(19) United States
(12) Patent Application Publication
(10) Pub. No.: US 2006/0204594 A1
(43) Pub. Date: Sep. 14, 2006

(54) Inhibition of enzyme activity by adsorptive removal of controlling ions

(76) Inventors: Blanka Taborsky Barnett, Fuqua-Varina, NC (US); Jiri Taborsky, Palmetto, FL (US)

Correspondence Address: Charles A. McClure, P.O. Box 1369, Lexington, VA, 24450, US

(21) Appl. No.: 11/410,870

(22) Filed: Apr. 26, 2006

Related U.S. Application Data
(63) Continuation-in-part of application No. 10/021,365, filed on Dec. 12, 2001.

(51) Int. Cl. A61K 33/12; A61K 33/06

(52) U.S. Cl. 424/683; 424/684

(57) ABSTRACT

Methods of preparing and using natural or synthetic zeolitic compositions therapeutically, so as to alleviate, cure, or even preclude human host disease attributable to exposure to bacteria within the Bacillus anthracis group: comprising, B. anthracis, B. cereus, B. Mycoide, and B. thurigiensis. Human exposure to the first member of the group causes the usually fatal disease, anthrax, in the absence of such effective pretreatment.

This application is a continuation-in-part of Ser. No. 10/021/365.

### TECHNICAL FIELD

This invention relates to inhibition of enzymes, especially to inhibition of microbial enzymes, and enzymes catalyzing development of neoplasms and metabolic dysfunctions, via interrelations of enzymes, ions, and ion exchanging compositions and/or ion adsorbents.

### BACKGROUND OF THE INVENTION

As stated by Bohinski [1], in Modern Concepts in Biochemistry, “the totality of cellular activity is intimately dependent on the type and concentration of ionic materials within the cell, both of which are subject to change by alterations in the extracellular environment.” As stated by Dressler and Potter [2] in Discovering Enzymes, “Not to put too fine a point on it, enzymes control all of the chemical transformations in the living world.”

Enzyme-controlled reactions are essential to all phenomena of life. Nearly every cellular activity is catalyzed by enzymes, many of the enzymes dependent upon associated cofactors. Though differences between those cofactors may not be sharp-edged, Holum's proposition [3] lists three generally accepted categories of cofactors (also see a schematic thereof in FIG. 1), thus:

a) a coenzyme: a non-protein organic substance (e.g., a vitamin) dialyzable, thermostable, and loosely attached to an apoenzyme; a true substrate for enzyme-catalyzed reaction, recycled in a later step of a metabolic pathway by another enzyme;

b) a prosthetic group: a dialyzable and thermostable organic substance, firmly attached to the protein of the apoenzyme portion; and

c) a metal cation activator, metal cations being critical to enzyme function, structure, and stability.

In general, the more complex an organism, the more complex and numerous its enzymes, and the more likely it can survive some enzymatic irregularity, such as inadequate concentration, or absence of a given enzyme. Whereas metabolism in vertebrates depends upon a vast number of enzymes, whose activity may require presence of other enzymes, coenzymes, or similar cofactors, more primitive life forms (e.g., viruses, bacteria, protozoa, fungi) survive with fewer enzymes, often controlled only by an ion activator, and may have their metabolism or replication terminated by dysfunction of a single enzyme. The present invention views ions, which activate or otherwise control activity or stability enzymes as targets, with objectives to deactivate, inhibit, or destabilize enzymes, and thereby to neutralize pathogens, control development of neoplasms, and undesirable metabolic processes—with minimal collateral damage to their respective hosts.

Current methods of inhibiting microbial enzymes rely mostly upon activity of chemical agents; limited in degree upon such other means as heat treatment, radiation, immunization, hormone application, or genetic engineering. However, all of these approaches often have severe limitations and/or serious side effects. Researchers focus upon chemical enzyme inhibitors, usually antibiotics or other chemicals, administered to the host organism, and often causing eventual deleterious side effects. Some of them interfere with cell division, and often are toxic to both host and invader, while many of them may contribute to development of resistant mutation of targeted and/or non-targeted pathogens.

The present invention directs attention to adsorption of ions necessary for biocatalysis, via ion exchangers. Since the vast majority of enzymes, for practical purposes of present invention, are enzymes activated or otherwise controlled by cations, the main attention is directed to the enzyme inhibition by cation exchangers such as hydrous aluminosilicate compositions or synthetic cation exchangers, here exemplified specifically by zeolites. However, all processes and methods for inhibition of metalloenzymes, and for the preparation and modification of cation exchangers—as disclosed in this invention—are analogically applicable for inhibition of anion-dependent enzymes, and for the preparation and modification of anion exchangers, as well as organic ionexchanger such The more detailed disclosure of zeolitic inhibition of enzymes is presented by way of example rather than limitation.

Both natural and synthetic zeolites are well known as adsorbents, carriers and ion exchangers of ionic substances often intended to catalyze or to inhibit certain chemical activity. Sometimes zeolites are used, either solitary or distributed within an organic polymer, to convey a toxin, a chelate, or a heavy metal cation as a bactericide or fungicide, as in cosmetics and medicines. See, for example, Yoshimoto et al. U.S. Pat. No. 4,870,107 (1989); Hagiwara et al. U.S. Pat. No. 4,775,585 (1988), U.S. Pat. No. 4,911,898 (1990), U.S. Pat. No. 4,959,268 (1990), Satoshi et al. Japanese Patent Application 03218916 A (1991); Satoshi et al. Japanese Patent Application 03255010A (1991); Wagner U.S. Pat. No. 4,824,661 (1989); and Barry U.S. Pat. No. 6,365,130 B1 (2002). In Chu et al. U.S. Pat. No. 5,140,949 (1992), for example, a mixture of zeolite and clay is proposed as a feed supplement, and as a topical treatment, based on its ability to adsorb ammonium cations. Similarly, in Polak et al. U.S. Pat. No. 5,409,903 (1995), zeolite alone, or zeolite in a mixture of other chemicals, is proposed for the treatment of Helicobacter pylori and dermatitis. U.S. Food Additive Regulation 582-2727 approves zeolite use in feeds as an anti-caking agent, and USDA approves them in food processing applications; being in EPA compliance (40 CFR, Part 180.1001 and elsewhere). Engler U.S. Pat. No. 5,900,258 features silicates, de-aluminated but neither deionized nor homoionized, to inhibit microorganism growth on and within textile and other interstitial or porous materials, also on relatively impervious extensive structural or working surfaces, and in nutrient material fed to chickens in order to evaluate its possibility for reducing incidence of microorganisms in or arising from such feed. All of the foregoing efforts are of minor interest. Other specific uses of zeolites as carriers of substances harmful to biological, sometimes enzyme-dependent, activity also could be cited, but also are distinct from the present invention.

### SUMMARY OF THE INVENTION

A primary object of the present invention is inhibition of enzymes, via adsorptive removal of their ions serving either as catalytic cofactors or as structural stabilizers or both, by ion exchangers.

Another object of the present invention is an adsorptive removal of ions from the immediate environment of targeted enzymes, thus preventing microbes and neoplasms from utilizing them for replenishment or for production of new enzymes.

A further object is to extend the present invention as a different approach to inhibition of enzymes in areas of medicine, cosmetics, dentistry, agriculture, and food processing.

One more object is to provide an alternative to antibiotics.

Yet another object of this invention is to inhibit any biotype, serotype or other induced or spontaneous mutation of microbes, including drug-resistant strains.

An additional object of the invention is to deactivate proteinaceous biotoxins (e.g., snake and insect toxins)—an objective that cannot be achieved by antibiotics.

Another object of this invention is to provide effective means of prophylaxis, to limit likelihood of infection from contaminated air, liquids, foodstuffs, bodily surface contact, etc.

A still further object is to accomplish the foregoing objects in an economically sound way and in a manner safe to the human organism.

In general, the objects of this invention are achieved by inhibiting activity of microbial and neoplastic enzymes, enzymes causing metabolic dysfunctions, and proteinaceous biotoxins, by supplying to the site of that activity an ion exchanger—for example a properly constituted aluminosilicate—effective to adsorb ions and related substances provocative of such undesirable biochemical activity.

By a synergic action, aluminosilicates appropriately selected, such as to density and size of pores, are adapted to serve as molecular sieves to bind entire specific molecules, e.g., toxins. This ability of suitable aluminosilicates is a practical expedient often resorted to in the substance-separation industries.

Alteration in relative affinities of natural zeolites for given monovalent and divalent cations, by dry heating pretreatment, and the benefits of doing so are disclosed in Taborsky U.S. Pat. Nos. 5,082,813; 5,162,276; and 5,304,365. Zeolites or equivalent compositions may be ion-pretreated, e.g., by deionization, or by homoionization, or may be synthesized in specific (e.g., hydrogen) cation form, and be applied as a broad-range adsorbent, or may be selectively reionized for specific applications. Equivalent compositions may be combined for complementary and/or synergic purposes and/or for their affinities for ions or classes thereof.

Other objects of the present invention, together with means and methods for attaining the various objects, will become readily apparent from the following description, presented by way of example rather than limitation.

### SUMMARY OF THE DRAWINGS

FIG. 1 comprises three schematized representations of a complete enzyme, comprising an apoenzyme (A) in separate conjunction with each of several different cofactors: (a, b, c).

FIG. 2 is a schematized representation of a zeolite (Z), in conjunction with each of a zinc activated protease (DP) and a tripartite toxin (LF+OF+PA) as in a digestive enzyme.

### DESCRIPTION OF THE INVENTION

The invention is characterized in practical terms, so as to enable its successful practice, regardless of any academic or theoretical conceptualization expressed in this exposition thereof, as concurrence in the latter is not a prerequisite for successful practice of the actual invention.

Whereas “ionization” generally means a process of producing ions, in this description “ionization” and its inflected forms (e.g., reionization, homoionization) have the meaning of charging or loading an ion exchanger with ions—as a logical opposite of the unambiguous term “deionization” (being a conventional term for removing ions). The term “adsorbent” means an ionexchanger with most of its ion exchangeable sites unoccupied.

For all practical purposes, principles and methods of enzyme inhibition of bacteria, viruses, neoplasms, and metabolic dysfunctions via adsorption of their activating, stabilizing, or otherwise controlling ions by ion exchangers are identical. However, for demonstration of such inhibition bacteria are most suitable, as in the instant example of bacteria of the Bacillus anthracis group.

Preliminary Testing of inhibition of enzyme activity by zeolitic adsorption was conducted using the accepted ninhydrin (1,2,3-triketohydrindene hydrate) test for presence of amino acids from enzymatic breakdown of casein.

1. At room temperature, 100 mg of bacterial protease was stirred into 100 ml of distilled water containing 10 g of deionized (method 2Bd) clinoptilolite particles (<74 μm). After about 10 minutes of mild agitation, 10 ml of this solution was stirred thoroughly into 50 ml of 5% DIFCO isoelectric casein solution, which tested negatively as to free amino acids an hour thereafter. This test indicated that the bacterial protease was deactivated, or the activating and/or stabilizing cations were depleted from the casein solution, or both.

2. To avoid possible confusion by an eventual interaction between casein and zeolite, the test was modified as follows: at room temperature, 100 mg of bacterial protease was stirred into 100 ml distilled water containing 10 g of deionized (method 2Bd) clinoptilolite particles (300>600 μm). After about 10 minutes of mild agitation, the suspension was filtered through a 200 μm nylon sieve, then 10 ml of the filtrate was stirred thoroughly into 50 ml of 5% DIFCO isoelectric casein solution, which then tested negatively for free amino acids an hour thereafter. This result indicated that the bacterial protease was deactivated.

3. Test 2 was then conducted in a more refined process by circulation of the therein specified solution in 10× larger volume through a bed of clinoptilolite particles (300>600 μm). It should be noted that some enzyme species are able to replenish their needed ions from their environment within some limited time after deactivation, and therefore, the time lapse before inoculation should be adjusted accordingly.

The zeolite adsorbed cations from the enzyme and inhibited it from breaking the casein down and providing amino acids for detection. Similar tests were conducted with different species of zeolite (phillipsite, chabazite), with deionized and H⁺ homoionized samples from different sources, and with synthetic zeolites (Y, Beta, and ZMS-5 powders), and all test results indicated inhibition of the protease.

Further analogous tests indicated that all previously used deionized and H⁺ homoionized natural and synthetic zeolites inhibited all tested bacteria, but tests with virgin natural zeolites were not entirely conclusive, suggesting a need for pretesting of each batch thereof, or for limiting actual operations to use of aluminosilicates pretreated as specified here, operative both in vitro and in vivo, such as for agriculture, dentistry, medicine, and biological instances generally.

Analogous tests to inhibit the (Cl⁻) anion-activated α-amylase by an anion exchanger (resin A-SIP OH) indicated positive results. However, a cation exchanger can achieve the inhibition of the same enzyme also via adsorption of Ca⁺⁺, which is necessary for stability of α-amylase.

Selection, Modification, and Applicability of Aluminosilicates.

The aluminosilicates, especially zeolites are customarily used as ion exchange media, in effecting separation and recovery of dissolved materials, liquids and gases, as carriers of ions. and specifically as molecular sieves (e.g. for cracking petroleum fractions). Aluminosilicate minerals occur in many geographical locations and include prominently zeolites: clinoptilolite, chabazite, phillipsite, analcite, brewsterite, faujasite, ferrierite, flakite, gmelinite, leucite, stilbite, and yugawaralite; also the layer (or pseudo-layer) silicates, vermiculites and smectites—often called layered clays; bentonites, and kaolinites.

The foregoing natural minerals are hydrated mixed aluminosilicates, with compositions determined largely by the constituents available when they were formed, resulting in diverse crystalline structures. Synthetic zeolites have been produced with more controlled compositions, and often are designated by a letter (e.g., “F”, “X”) appended to “zeolite.”Whether produced under laboratory conditions or in mineral deposits, these ion exchangers range widely in composition and physical properties. Their identification, as well as their properties, can vary, depending upon specific interesting characteristics—here, their physical properties, modification and manipulation of accessible surfaces and sites for adsorption of cations.

Ion exchangeable aluminosilicates have a distinctive molecular arrangement causing a negative charge of their molecules. It results in strong adsorptive power, unmatched by any other adsorbent and strong enough to penetrate the protective coats of vegetative forms of microbes, and moist-swollen exosporia, and protective coats of endospores. A similar transfer of cations through gels has been well demonstrated (see section 7B below: Application of aluminosilicate enzyme inhibitor). Aluminosilicates form extremely porous crystalline structure having tiny uniform pores, measuring in some species only a few Å, and endowing them with tremendous interior surface area having numerous ion exchangeable sites. Their negative charge enables these sites attract, adsorb, and eventually exchange, cations.

Consequently, aluminosilicates possess a unique ability to adsorb metallic activators of enzymes controlling biochemical processes in viruses, bacteria and some other low-organized organisms. Natural aluminosilicates, synthetic zeolites and other ion exchangers were tested in practicing and evaluating this invention.

Natural aluminosilicates or synthesized zeolites, when properly selected and modified, are, for all practical purposes, chemically inert and do not cause any chemical side effect to the host organism. They are not recognized by the pathogen or by the host as xenobiotics. Therefore, they are unlikely to trigger any immunologic reaction in the host or to activate any defense mechanism of the pathogen. Hence, the pathogens are unlikely to develop any resistance to the loss of enzyme activator, as in the practice of the present invention.

As therapeutics and prophylactics, aluminosilicates work in three principal ways, without any appreciable toxic or biochemical impact on the host organism:

a) inhibiting activity of microbial and neoplastic metalloenzymes;

(b) deactivating toxins; and

(c) adsorbing cations from immediate microenvironments, thus preventing microbes and neoplasms from utilizing them for replenishment or for production of new metalloenzymes.

(d) in synergic effect as microbial enzyme inhibitors and desiccants, they are extremely useful in dermatology for topical therapy of wet wounds, blisters, non-healing wounds, ulcers, eczemas, skin cancers, herpes blistering, etc.

Virgin aluminosilicates exhibit substantial differences in chemical composition, crystalline structure, density, and levels of impurities. Aluminosilicates of high density, aluminosilicates with a considerable crystalline silica contamination, fibrous aluminosilicates, and aluminosilicates contaminated with specific cations, and the like, are unsuitable for medical or pharmaceutical purposes. Accordingly, deionized, homoionized, selectively ion-recharged, or otherwise modified natural aluminosilicates and synthetic zeolites are preferable to virgin aluminosilicates in the practice of this invention.

1. Typical Ion Exchangers Used in Experiments:

A. Natural zeolite: clinoptilolite, hydrated sodium potassium calcium aluminum silicate (Na, K, Ca)2O.Al₂O₃.10SiO₂.8H₂O), Winston, N.M. deposit, 4×6 size granules (approx. 5 mm).

Analysis (weight % for major oxides): Bowie and Barker, NM Bureau of Mines, 1986): Silicate 64.7%, CaO 3.3%, MnO 0.1%, Al₂O₃ 12.6%, MgO 1.0%, TiO₂ 0.2%, K₂0 3.3%, Fe₂O₃ 1.8%, and Na₂0 0.9%.

Chemical Composition for given elements, by x-ray fluorescence (ppm, or wt. % noted; by Desborough, USGS OF Rpt 96-065 & 265, 1996.):

| | | | | | | | | | |
|---|---|---|---|---|---|---|---|---|---|
| K | 2.0% | Cu | 30 | Zr | 190 | Nd | 15 | Ca | 2.7% |
| Fe | 0.9% | Rb | 70 | Nb | 20 | Ba | 1030 | Sr | 1720 |
| Ce | 90 | Pb | 40 | | | | | | |

Cation Exchange Capacity:

1.00-2.20 meq/g (may vary, as CEC values are relative to procedure and specific cations).

Major Exchangeable Cations: Rb, Li, K, Cs, NH4, Na, Ag, Ca, Cd, Pb, Zn, Ba, Sr, Cu, Hg, Mg, Fe, Co, Al, Cr, Mn, H.

(Selectivity of such cations is a function of hydrated molecular size and relative concentrations).

Purity:

Analysis by x-ray diffraction at the N. M. Institute of Mining and Technology and other tests suggest an 80% clinoptilolite content with the remaining material primarily inert volcanic ash and sediments. Clay and other mineral varieties are detectable only in minute quantities.

Physical Properties:

| | |
|---|---|
| pH (natural) | 8.0 (approx.) |
| Acid Stability | 0-7 pH |
| Alkali Stability | 7-13 pH |
| Bulk Density (dried, −40 Mesh) | 783-1054 kg/m³ |
| Cation Exchange Capacity (CEC) | 1.0-2.2 meq/g |
| Color | White (85 optical reflectance) |
| Crushing Strength | 2500 lbs/in³ (176 kg/m³) |
| Hardness | 3.5-4.0 Mohs |
| LA Wear (Abrasion index) | 24 |
| Mole Ratio | 5.1 (SiO₂/Al₂O₃) |
| Other | non-soluble, non-slaking, free |
| | flowing |
| Pore Size (diameter) | 4.0 Å |
| Pore Volume | 52% (max.) |
| Resistivity | 9,000 (approx.) ohms/cm |
| Specific Gravity | 2.2-2.4 |
| Surface Area | 1357 yd²/oz (40 m²/g) |
| Swelling index | 0 |
| Thermal Stability | 1202° F. (650° C.) |

B. Synthetic zeolite: Zeolyst® Y Type zeolite powder (FAU) CBV 400 in cation form.

| | |
|---|---|
| Molecular Ratio | 5.1 (SiO₂/Al₂O₃) |
| Unit Cell Size | 24.50 Å |
| Surface area | 730 m²/g |

C. Bead Cellulose: Perloza® MT 50, a macroporous gel bead cellulose, stabilized by 25% ethanol.

| | |
|---|---|
| Particle size | 100-250 μm |
| Temp. resistance (wet/pH 7.0/1 hr) | 120° C. |
| Stability within pH range | 1-14 |
| Stability in salt solutions | with ionic strength up to 10 mol/l |
| Chemical resistance | aqueous solutions, buffer, organics, |
| | detergents, and chaotropic agents |
| Swelling in aqueous solutions | max 1 vol % |

For purposes of this invention, the suitability of bead cellulose and its derivates is limited. They must withstand a wet stage, because the process of desiccation severely damages their porosity.

D. Anion exchanger: Anion Resin in Hydroxyl Form (A-S1P OH)

2. Modification of Aluminosilicate Cation Exchangers:

A. H⁺ Homoionization:

a. H⁺ Homoionization Via Ammonia Decomposition:

The aluminosilicate is first impregnated by ammonium cations to displace the achievable maximum of other cations via ion exchange, then washed, dried, and finally heated to 500° C. At this temperature, ammonium decomposes to gaseous ammonia, which escapes, and hydrogen cations, which occupy available adsorption sites of the aluminosilicate. The thermal stability of treated aluminosilicate species and types must be considered unless a distortion of crystalline structure is negligible for the given application, or if a certain distortion of the crystalline structure is desirable as an additional functional modification.

b. H⁺ Homoionization Via Electrolysis of Water:

A stream of hydrogen cations generated by electrolysis of water is directed through a bed, preferably a fluid bed, of sand or granule-sized aluminosilicate. Via ion exchange, an achievable maximum of other cations on ion exchangeable sites is replaced by hydrogen cations. The excess of hydrogen cations is reduced on the cathode to hydrogen gas. The cathode should be placed in a trapping device that collects reduction products and positively charged impurities. While this is an elegant and very pure method, an eventually insufficient concentration of electrolyte may render the H⁺ homoionization imperfect. However, this is the only practical method of H⁺ homoionization of hydrocolloid aluminosilicates.

The performance of the process may be improved by a modification of the electrolytic apparatus, provided by rotating chambers, by rotating phases of polarity, and/or by enhancing the electrolyte with ionized effluent water from one (or more) auxiliary electrolyzer(s). Such modified electrolytic apparatus also may be applied advantageously for ionization, homoionization, or reionization processes described later.

c. H⁺ Homoionization Via Acid Treatment:

Most acids are suitable, but inorganic acids are preferable, especially nitric acid because of easy and environmentally sound disposal of NO₃⁻ anion effluent. However, for purposes of some special cation exchanger's selectivity, the use of organic acids must be considered. Adsorbent of sand or granule size is treated with diluted (e.g., 3%) acid in order to displace the achievable maximum of other cations by hydrogen cations via ion exchange, then rinsed with redistilled or medical grade deionized water to remove formed salts and anions to an achievable minimum.

Unless a synergic low pH effect is sought, the pH of H⁺ homoionized adsorbents should be adjusted by washing with redistilled or medical grade deionized water, or by OH⁻ treatment to establish the desirable pH value.

B. Deionization:

a. Partial:

The adsorbent is washed with redistilled or medical grade deionized water until most ion exchangeable sites are, by equilibration, free of cations. For achieving high degree of deionization, this method is too time consuming and economically unfeasible.

b. By Water Electrolysis:

A bed, preferably a fluid bed, of adsorbent is first electrolytically H⁺ homoionized (method 2Ab), rinsed, and then the polarity of electrodes is reversed, whereby the aluminosilicate bed is exposed to a stream of hydroxyl anions (OH⁻) until the desired pH is stabilized.

c. By a Combination Method:

A bed, preferably a fluid bed, of adsorbent, already H⁺ homoionized (method 2Aa, 2Ac) is exposed to a stream of hydroxyl anions (OH⁻) until the desired pH is stabilized.

d. By (OH⁻) Effluent from an Anion Exchanger:

A bed, preferably a fluid bed, of H⁺ homoionized adsorbent is exposed to an OH⁻ water effluent generated by an anion exchanger until the achievable maximum of hydrogen cations has been removed from the aluminosilicate (or until a desired pH is established), via formation of water during the process of equilibration.

C. Selective Ionization:

For specific purposes (e.g., to prevent or to mitigate adsorption of selected cations, such as calcium or iron cations, or to deliver to the site cations for specific purposes (e.g., copper or cobalt ions), adsorbents may be ionized with any selected metal cation or cations via appropriate salts or hydroxides. A selective ionization may be implemented during or immediately after homoionization, or instead of homoionization. The following methods are preferred in the practice of this invention.

a. Selective Reionization by Cations Toxic to Microbes or Neoplastic Growth:

Using untreated (virgin) zeolite as a carrier of toxic cations to function at a destination site is known. However, for medical purposes the prior art is generally unsuitable because the concentration of such toxic cations in virgin aluminosilicate is difficult to establish and maintain because of uncontrollable factors, such as impurities, present unavoidable cations, pH fluctuation and consequent fluctuation of toxicity level, and equilibria in the microenvironment and within the aluminosilicate. In many applications accuracy in the cation concentration is critical. For example, excessive concentrations of copper cations will mitigate or completely inhibit production of mucous surfactant, which protects the host's gas-exchanging cells [4][5], and thereby may cause irreversible damage to a host's respiratory system and eventually result in death of the host. The methods of pretreatment of aluminosilicates as disclosed in this application, especially in applications of deionized aluminosilicates, allow appropriate accuracy of the dosage of toxic cations, thereby protecting the host's tissues, and equalizing any eventual site competition.

b. Selective Ionization of Adsorbent by Auxiliary Cations

This is of special interest. For example, one of the severe symptoms of inhalational anthrax is shortness of breath. It is caused (besides the bacterial damage to the alveolar epithelium) by the consumption of zinc ions by anthrax bacilli. Zinc cation is the primary activator of carbon anhydrase—the enzyme catalyzing the reversible hydration of CO₂ to H₂CO₃, a necessary reaction for facilitation of transport of CO₂, and transfer and accumulation of H⁺ and HCO₃⁻. Hence, the deficiency of zinc cations contributes substantially to the inhibition of the respiratory gas exchange process. However, some carbon anhydrases are able to function with an alternative metal activator, as with the cobalt cation in this instance [19], and possibly with other metal cations, e.g., cadmium. Ion-exchanger adsorbing zinc activators from bacteria, from their immediate microenvironment, and from their toxins, can serve simultaneously as carriers of cobalt cations to boost the carbon anhydrase catalytic activity, and thereby greatly mitigate the “short breath” symptoms. The cobalt cations may be administered via an ion-exchangeable carrier, or as a part of a compound (e.g., salt or chelate) in any suitable therapeutic form (e.g., aerosol, hydrosol, intravenous infusion, or extracorporeal filtration of bodily fluids through a bed of cobalt-impregnated ionexchanger).

3. Life Forms Used for Testing of Enzyme Inhibition; Experiments, Observations, and Evaluations:

A. Bacteria of the Bacillus anthracis Group:

Within the genus taxon, the current taxonomic and nomenclatural rules do not recognize any “group” taxon—which is only ancillary, indicating a close systematic and phylogenetic relation of certain species, here those of B. anthracis, namely: B. cereus, B. mycoides and B. thuringiensis. This group is frequently informally designated as the Bacillus cereus group. See, Genus Bacillus Cohn 1872. Hierarchy: Monera Bacteria-Inside series of Bacteria-Bacillales. Nomenclatorial/taxonomic status: Approved Lists Type species: B. subtilis Reference(s): Int. J. Syst. Bacteriol. 30:256 (AL), (Bergey's manual of determinative Bacteriology, 8th ed., 1974; Editors: Buchanan, R. E., Gibbons, N. E; Publisher: The Williams & Wilkins Co., Baltimore).

The B. anthracis group is a group of closely related species within the genus Bacillus. Though classified as valid different species, these organisms seem to differ only in the plasmids. All four species are large straight rod-shaped Gram-positive, non-flagellated, endospore-producing bacteria, whose spores do not swell the sporangium. They are often aerobic cells of 1-10 μm in length, and 1-1.5 μm in breadth, with a “jointed bamboo-rod” cellular appearance. All species of the B. anthracis group are pathogenic to humans, causing known or potential cutaneous/subcutaneous, intestinal, inhalational and other infectious conditions. The endospores are approximately 1 μm, species-indistinguishable within the group. Endospores are extremely resistant and may survive, for entire geological periods, at temperatures ranging from absolute zero to −40° C., and for decades between −30° C. and at least 50° C. They can withstand several minutes of usual autoclave sterilization and at least one minute of usual microwave sterilization. They germinate readily, and their vegetative cells grow on all ordinary laboratory media, at like temperatures and times, except that B. anthracis prefers a range closely about 37° C. Bacteria of the B. anthracis group share a multitude of other characteristics, including both biochemical and biophysical properties. Differentiation of the respective organisms is done in the vegetative form by determination of motility (B. cereus rods are usually motile), and by the presence of toxin crystals (B. thuringiensis), and also by hemolytic activity (B. cereus and B. thuringiensis are beta-hemolytic, B. anthracis is usually non-hemolytic), by growth requirement for thiamin, by lysis via gamma phage, by growth on chloral-hydrate agar, and further by the morphology of micro-colonies (e.g., a rhizoid growth is characteristic for B. mycoides, and a perloid growth pattern for B. anthracis).

A. Bacillus anthracis (Cohn 1900), various synonyms: Bacillus cereus var. anthracis (Cohn 1872); Smith et al. 1946; Bacteridium anthracis (Cohn 1872); Hauduroy et al. 1953. Nomenclatorial/taxonomic status: Approved Lists Reference(s): Int. J. Syst. Bacteriol. 30:256 21 (AL), Ref.: Bergey's manual of determinative Bacteriology, 8th ed., 1974; Editors: Buchanan, R. 22 E., Gibbons, N. E; Publisher: The Williams & Wilkins Co., Baltimore); Risk group: 3 (German 23 classification) Type strain: ATCC 14578. Bacterial proteolytic enzyme: Zn⁺ activated protease; 24 LF of the tripartite toxin: specific Zn⁺⁺ activated protease; OF: adenylate cyclase.

Bacillus anthracis is usually an aerobic, nonmotile species. The vegetative cells are large rods (1-8 μm long, 1-1.5 μm wide). B. anthracis is the causative agent of the anthrax disease. The symptoms of all three forms (cutaneous, intestinal, and inhalational) are well known [15]. Anthrax has been intended to be the most dangerous biological warfare agents for more than eighty years. Within that time, countless deadly strains have been developed, many of them as antibiotic-resistant and drug-resistant strains. Neither trials nor any cultivation of B. anthracis were conducted for purposes of this invention, but experimentation on other of the members of the group has been undertaken successfully and tentatively is believed to be applicable to every B. anthracis group member, based upon their close phylogenetic relationship.

B. Bacillus cereus (Frankland & Frankland 1887) ambiguous synonym(s): Bacillus cereus var. anthracis, Bacillus thuringiensis, Bacillus endorhythmos, Bacillus medusa. Nomenclatorial/taxonomic status: Approved Lists Reference(s): Int. J. Syst. Bacteriol. 30:256 (AL), (Ref: Bergey's manual of determinative Bacteriology, 8th ed., 1974; Editors: Buchanan, R. E., Gibbons, N. E; Publisher: The Williams & Wilkins Co., Baltimore); Risk group: 2 (German classification) Type strain: ATCC 14579, CCM 2010, NCm 9373, NCTC 2599. Bacterial proteolytic enzyme: Zn⁺⁺ activated protease; LF of the tripartite toxin: specific Zn activated protease; OF: adenylate cyclase. Intestinal infection, causing food poisoning, has been believed for a long time to be the only medical concern. The symptoms of the diarrhea type of food poisoning mimic those of Clostridium perfringens, beginning with watery diarrhea, sometimes accompanied by nausea and vomiting. Abdominal pain and cramps occur 6-15 hours after infection. Usually such symptoms persist for 24 hours. Symptoms of the emetic type are similar to those of Staphylococcus aureus: nausea and vomiting within 30 minutes to 6 hours after consumption of contaminated food. Abdominal cramps and diarrhea may occur too. Symptoms generally last less than 24 hours.

Recently, however, cutaneous B. cereus infections causing acute necrosis very similar to the cutaneous form of anthrax have been reported. Even more dangerously, several cases of B. cereus infections of other tissues occurred: including rapidly fatal meningoencephalitis [14], septicemia, mastitis, and several cases of potentially blinding endophthalmitis [6][7].

Since B. cereus is a typical airborne-spore proliferater, and sporulates and germinates easily, it is a potential agent for inhalation infections. No verified case of an inhalational form of infection has been reported yet. It may be hypothesized that B. cereus OF enzyme did not mutate yet—as B. anthracis did—to be effective enough to impair the host's defense system. Inocula: Bacillus cereus strain CBSC 15-4870/2001, freeze-dried CBSC 15-4870A/2001.

C. Bacillus mycoides (Fluegge 1886), ambiguous synonym: Bacillus mycoides corallinus. Hefferan 1904. Nomenclatural/taxonomic status: Approved Lists Reference(s): Int. J. Syst. Bacteriol. 30:257 (AL), (Ref.: Bergey's manual of determinative Bacteriology, eighth ed., 1974. Editors: Buchanan, R. E., Gibbons, N. E; Publisher: The Williams & Wilkins Co., Baltimore; Die Mikroorganismen, 3rd ed. vol. 2, 1896; Editor: Fliigge, C.; Publisher Vogel, Leipzig); Risk group: 1 (German classification); Type strain: ATCC 6A62. Bacillus mycoides is in almost all of its characteristics like B. cereus—but for its morphological rhizoid pattern of micro colonies. Bacterial proteolytic enzyme: Zn⁺⁺ activated protease; LF of tripartite toxin: specific Zn⁺⁺ activated protease; OF: adenylate cyclase. Inoculum: B. mycoides strain CBSC 15-4870/2001.

D. Bacillus thuringiensis (Berliner 191+5) ambiguous synonym: Bacillus cereus var. thuringiensis (Berliner 1915) ambiguous synonym: Bacillus cereus var, thuringiensis (Smith et al. 1952). Nomenclatural/taxonomic status: Approved Lists Reference(s): Int. J. Syst. Bacteriol. 30:258 (AL) (Ref.: Bergey's manual of determinative Bacteriology, 8th ed., 1974; Editors: Buchanan, R. E., Gibbons; N. E; Publisher: The Williams & Wilkins Co., Baltimore); Risk group: 1 (German classification); Type strain: ATCC 10792, Sp2000 Taxon Code: BIO-6867. Bacterial proteolytic enzyme: Zn⁺⁺ activated protease; LF of the toxin: specific Zn⁺⁺ activated protease; OF: adenylate cyclase.

B. thuringiensis is a bacterium, marketed worldwide as a specifically targeting bioinsecticide for control of plant pests (mainly caterpillars of the Lepidoptera), for control of mosquito larvae, simuliid blackflies, etc. Genetic material from B. thuringiensis toxin is used in the development of genetically engineered corn, cotton, and other crop plants. Most BT insecticides are derived from genetically improved mutations of B. thuringiensis biovar israelensis or B. thuringiensis kurstald. The active ingredients of marketed BT products are the bacterial dormant spores (>1012 per liter) and proteinaceous aggregates, including crystal-like parasporal inclusion bodies (PIB). The research done for manufacturers of BT products presents the bacterium as safe to human health. Yet, much as may be indicated, for example in DiPel®DF MSDS [16], the trials appear purpose-designed. In general, the health implications of exposures to B. thuringiensis, especially inhalational effects, have not been yet satisfactorily investigated.

Because of the close phylogenetic relation between B. anthracis group species, it should be taken into consideration that a dose of Bt spores, sufficiently potent to cause an inhalational Bt infection, may cause an infection with symptoms mimicking the symptoms of an anthrax infection (in at least one test, the mortality in guinea pigs was 10% [10]).

The BT products generate nonspecific cytotoxicities involving loss in bioreduction, cell rounding, blebbing and detachment, degradation of immuno-detectable proteins, and cytolysis. Some research data indicate that spore-containing BT products have an inherent capacity to lyse human cells in free and interactive forms and may also act as immune sanitizers [17]. Inocula used: Bacillus thuringiensis strain CBSC 15-4870/2001 (vegetative cells), CBSC 154870A/2001 (lyophilized vegetative cells), Javelin® (endospores, strain not identified), Thuricide® (endospores, strain not identified), and Skeetal® Abate (endospores, strain not identified).

Classified in the Bacillus anthracis group may be a newly described species Bacillus pseudomycoides but there has not yet been sufficient research done to validate it.

Bacillus pseudomycoides Nakamura 1998 Reference(s): BIO-6840, Nakamura (L.K.): Bacillus pseudomycoides sp. nov., Int. J. Syst. Bacteriol., 1998, 48, 1031-1035. No trial nor any cultivation of B. pseudomycoides has been conducted for the purposes of this invention. Many characteristics of B. anthracis, B. cereus, B. mycoides and B. thuringiensis are alike. For this invention the most important trait is that the bacterial metabolic protease and the lethal factor (LF) of the toxin are zinc-dependent; that is, the enzymes are activated by the zinc cation [8]][9][19][11][12][13]. Thus, it can be assumed with reasonable certainty that the mechanism of inhibition of their metabolic proteases and the deactivation of their toxin enzymes by adsorptive removal is similar in all four species, especially in B. cereus and B. anthracis, being so clearly alike in so many regards.

4. Culture Media

A multitude of suitable media for culturing Gram⁺ bacteria has been tried, including standard beef bouillon, nutrient gelatins and broths, count agar, modified nutrient agar (without peptone), protein-enriched nutrient agar, TSA w/5% and 10% sheep blood, etc. All of the media tested supported growth of vegetative cells and germination of endospores (where applied) along with the expected unimportant differences in morphological patterns of micro-colonies. After preliminary testing for suitable uniform media, a T-011 modified nutrient agar was chosen, consisting of standard beef extract, 5 g; agar, 15 g; with rehydration, 23 g/1000 ml. At its pH of 6.8, this agar is well within the optimal range for the Bacillus anthracis group.

5. Inoculation of Bacteria

Many inoculation methods were tried in preliminary assays, including direct swab smear, diluted smears, loop inoculations in varied cell concentrations, smears and loops of inocula diluted in redistilled water, as well as smear and loop inocula diluted in physiol. solution. Dry inoculates of spores were tried also (where applicable). In all trials, the temperature was maintained at approx. 25° C. All of these experimental methods proved satisfactory. After these preliminary trials, two specific methods were selected for formal experimentation, as follows:

A: a swab smear inoculum from an established culture, diluted in redistilled water in a 1:10 approx. wt. ratio (cell:water). Cell count was not done. A long, single smear was applied.

B: 0.5 ml of diluted inoculum just described above (5A) was spot-dropped in the center. Note: Dry spore inoculation was abandoned in the final trials because the resulting rapidity of germination would have required needlessly difficult measurement of very small time intervals.

6. Actual Experimental Results

The results in all trials proved positive, as expected in view of the preliminary trials. There were expected differences in vigor of the growths, in morphological patterns of micro-colonies, plus some aberrations from standard phenotype, but none pertinent to this invention. The following findings have been clearly established: A. the ionized zeolite inhibits Zn⁺⁺ activated bacterial proteases; and B. the inhibition is substantially instantaneous.

7. Application of Aluminosilicate Enzyme Inhibitor

In some preliminary trials, the inhibitor was applied after a growth of micro-colonies was apparent under 10× magnification. This method was abandoned after it was well established that the inhibitor has an instant effect. Such instant effect is illustrated in FIG. 2. Also in some trials with fluid media, a similar delay in inhibitor application was adopted.

A. For the inoculation method 5A, pH 6.4 stabilized deionized clinoptilolite particles of mesh 200 (<74 μm) were applied onto one half of the plate (the other half serves as a control) immediately after inoculation: a. as a dust, and b. as a hydrosol.

B. For inoculation method SA, pH 6.4 stabilized and deionized clinoptilolite particles of mesh 200 (<74 μm) were applied saturated in pieces of an inert porous absorptive material (filter paper) on the margin of the plate.

Note: In some preliminary runs, a bicomposite medium was also tried, by pouring one half of the plate in the original formulation, and the other half incorporating aluminosilicate inhibitor. Smearing inoculum over both halves of the plate, made the inhibitor effect immediately apparent.

The latter alternative also proved the tremendous adsorptive power of aluminosilicates to transfer ions through gel substances, as for example mucus, covering GEC's (gas-exchanging cells) in alveoli and elsewhere, or even more importantly, the protective coats of microbes.

8. Detailed Description of Drawing Figures

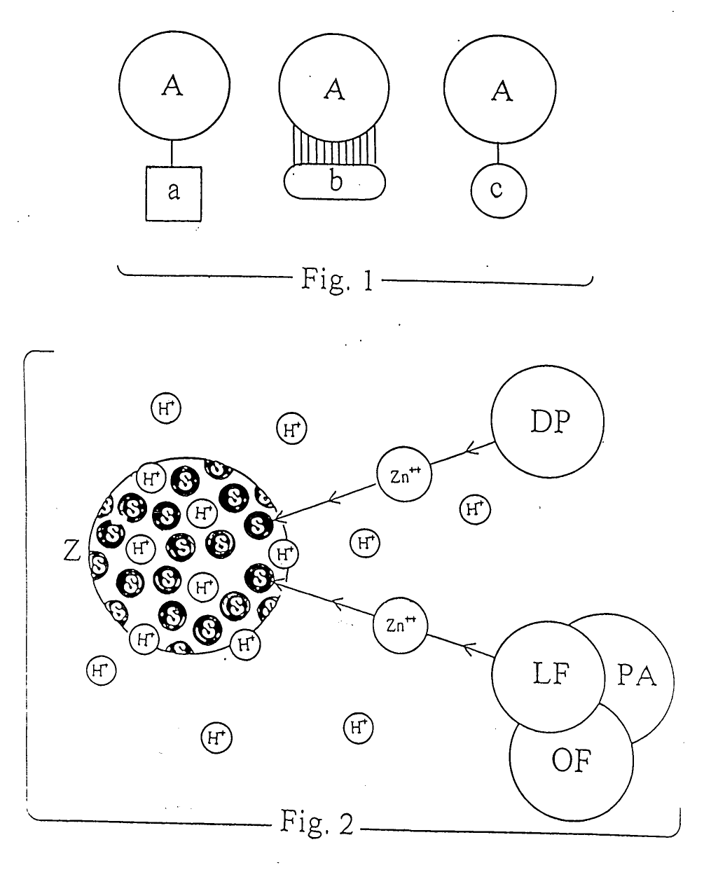

FIG. 1: Enzyme cofactors according to Holum

A complete enzyme (holoenzyme) consists of an apoenzyme (A) and one or more cofactors of the following three types: a, b, and c.

a. Coenzyme, a non-protein organic substance (e.g., a vitamin) which is dialyzable, thermostable, and loosely attached (single vertical connecting line) to the apoenzyme. It is a true substrate for enzyme-catalyzed reaction, and is recycled in a later step of a metabolic pathway by another enzyme.

b. Prosthetic group, a dialyzable and thermostable organic substance. It is more firmly attached (multiple linking vertical connecting lines) to the protein of the apoenzyme portion. Metal activator, a loosely attached metal cation, e.g., Zn⁺⁺, K⁺, Fe⁺⁺, Ca⁺⁺, Mg⁺⁺, Co⁺⁺, Cu⁺⁺, or Mn⁺⁺. The metal cations are critical to the enzyme function, structure, and/or stability, and they ultimately are .of great biological and medical importance.

FIG. 2: Adsorption of enzyme activator via zeolite (H⁺ concentration ˜10⁻⁷)

Hydrogen cations (H⁺) occupying some ion exchangeable sites (S) on the exosurfaces and endosurfaces of zeolite Z are in equilibrium with H⁺ cations of the surrounding environment. When a bacterial digestive enzyme, a zinc-activated protease (DP), or a tripartite toxin (comprising zinc-dependent lethal factor LF, plus oedema factor OF (an adenyl cyclase), plus four-domain protein PA, enters the adsorptive range of such a zeolite particle, zinc cations Zn⁺⁺ will be adsorbed, immediately inhibiting the bacterium and deactivating the toxin. Also deactivated will be toxins already excreted by the pathogens into the host's macroenvironment (e.g., epithelium of alveoli, or bodily fluids.)

The drawing area designated Z represents only a minuscule part of the adsorptive surface of a zeolite particle, with interconnecting porous crystalline structure. The small size of most aluminosilicate pores (e.g., 4 Å, in the clinoptilolite example herein) precludes entry of pathogens or their proteins (e.g., bacterial digestive proteins, or toxins), whereupon rapid adsorption occurs on the outer surface, and (upon reaching equilibria there) thereafter proceeds within the particle of the ion exchanger.

9. Mode of Inhibition

The negatively charged ion exchanger attracts and adsorbs the cation activators of enzymes [FIG. 2] which renders the enzymes (the bacterial digesting protease and toxin's LF protease) deactivated.

10. Practical Applications of Adsorbents:

In all applications, the ion adsorption is enhanced greatly in a wet environment. In a dry environment, e.g., on skin, inhibitor should be applied wet and be maintained wet (however, see 10.A.a.) For therapy of cutaneous and intestinal infections, the use of suitable aluminosilicates is entirely safe. For inhalational infection therapy, and in applications involving the circulatory system, a number of side effects of mechanical and biophysical nature should be considered.

A. Cutaneous infection and neoplasia: the adsorbent may be applied topically, as follows:

a. Dry: applicable in powder form directly to provide a synergic desiccating effect for wet or watery wounds, blisters, burns, herpes lesions, ulcers, bleeding wounds, etc.

b. Wet: for cutaneous infections and neoplasms not acutely wet, the adsorbent should be used preferably wet—mixed with clean water, preferably distilled water, and applied as a spray or a thin paste; incorporated in an inert gel; as a gel-like mixture with powdered hydrated layered clays, or in dressings or bandages impregnated with ion exchanging inhibitor.

B. Intestinal infection May be Treated, as Follows:

a. By ion adsorbent administered orally, preferably before a meal, mixed in a drink (water, milk, tea) devoid of any salt preservative. USDA approved addition of zeolite in feed is very conservative at 2%. A substantially higher dose (more than 5 times the approved rate) may cause a temporary depletion of intestinal flora. Particle size of the ion adsorbing inhibitor is not critical; 200 mesh (approx. 75 μm) being good. Of course, the smaller the particles are, the faster the adsorption process will be. Ion adsorbing inhibitor passing through the digestive system deactivates bacteria, viruses, protozoan, certain worms, toxins, and digestive metalloenzymes; then it is excreted.

b. Following ingestion, the adsorbent is at least partially H⁺ homoionized by the stomach acid, and then gradually stabilized to the equilibrium in the small intestine.

C. Inhalational Infection

Preparation of dry aluminosilicates in the very small particle size (<3 μm) necessary to reach the inner epithelium of alveoli is technically difficult. The most practical mode of administration is inhalation of a mist containing submicron particles, easily calibratable by sedimentation in a water column. Insofar as there is a concern about clogging of alveoli, as caused by an eventual extremely large overdose, even a complete coverage of the alveolar epithelium by adsorbent's particles delivered via aerosol (or hydrosol) of suitable composition (e.g., containing surfactant) would not inhibit in appreciable extent the functioning of the epithelium. The adsorbent's capacity for CO₂ adsorption is negligible, in view of the huge volume of CO₂ exchanged in the lungs. However, unlike an a hydrosol, aluminosilicate dust in high concentrations, as in emergency use when water for appropriate preparation is not available, may temporarily desiccate the alveolar surface and hence may cause an extended need—expressed as coughing and temporary feeling of shortness of breath—for production of alveolar surfactant, as by type II cells. Even though only partial and temporary, such an administration process should be carefully monitored.

b. Except in utmost emergency, intravenous application should be avoided. Some specific concerns are obvious, such as deposition of adsorbent's particles in tissues. For an intravenous application, the particle size of the adsorbent in the injected solution should be <1 μm, preferably close to the particle size in the colloidal suspension. In order to prevent an eventual calcium deficiency shock, or iron deficiency in hemoglobin, and similar cation-dependent problems, the deionized adsorbents should be recharged with selected cations (e.g. calcium, iron, or magnesium cations).

c. In a hospital or similar setting, blood can be filtered (outside the body) through a bed of adsorbent. This method is important in a situation when the toxins in blood reach an otherwise uncontrollable concentration. The particle size should be between 200-500 μm to allow free flow of blood and an instant effect, and be pretreated as described above in b. to prevent any eventual cation adsorption problems.

Also, adsorbents may be selectively reionized by cation(s), which interfere with biochemical and biophysical processes in a pathogen (e.g., Ag⁺) or, more specifically, interfere with production or maintenance of protective coats of pathogens (e.g., Cu⁺⁺).

11. Prophylaxis

A. Dry and wet filters for gas masks, emergency homemade masks, mass transportation and building air filtration systems, etc.

B. Decontamination of skin and hair, clothing, homes and other enclosed or open areas

C. Decontamination of drinking water and food, especially fruit and vegetables.

12. Other Bacteria Used:

Pseudomonas fluorescens, P. putida, Xanthomonas citri, B. brevi, Escherichia coli, Salmonella enteritidis, Citrobacter freundii, Enterobacter aerogenes, Enterococcus faecalis, Micrococcus luteus, Rhodospirillum rubrum, and Vibrio fisheri.

B. Viruses: All testing, experiments, observations, and evaluations of inhibition of viral enzymes were conducted in vivo, based on symptoms, under the generally accepted principle that viral metalloenzymes are practically the same in the molecular structure, and modus operandi of the metalloenzymes of higher life forms, especially bacteria.

Experiments upon Tobacco Mosaic Virus indicate that zeolites can be used as a universal viricide in agriculture (TMV affects almost 200 known genera of plants and causes serious damage in cultivated crops).

Primary leaves of twenty tomato seedlings were inoculated with TMV. Developed necrotic lesions were sprayed in parallel tests with a 10 g/liter water suspension (particles <75 μm) of virgin natural zeolites (chabazite and clinoptilolite), homoionized natural zeolites (chabazite and clinoptilolite), and synthetic Y, Beta, and ZMS-S zeolite powders. Spraying was repeated after 24 hours. With identical results in all tests, all lesions were dry within 2-3 days, and no new lesions developed in any of the treated plants. The tests also indicate a systemic effect of zeolites.

A suitable example is the apoenzyme of HIV protease, which is activated by zinc cations. It seems virtually impossible that a fundamental mutation would occur so as to enable the apoenzyme to be activated by some other source. If the ion changed, the same or other zeolites would be likely to adsorb it also.

In contrast, HIV protease inhibitors, such as saquinavir, ABT387, or ribavirin are known to be deactivated rapidly in the host by cytochrome P450 enzymes, so only a small fraction of the inhibitor encounters the virus. The host system has to “metabolize” most of the inhibitor drug, incurring severe side effects. To counter them, the protease inhibitor may be administered in combination with another drug, such as ritonavir, which suppresses cytochrome P450 enzymes, or steroids to prevent general immunologic overload It is highly desirable to identify inhibitors that operate directly upon viruses without likelihood of deactivation.

Notwithstanding that it is not known how the cytochrome enzymes identify the foreign chemicals, we can hypothesize with fair confidence that a zeolite, which does not react chemically and behaves—except for the adsorption—as an inert substance, will not be recognized by the cytochrome as xenobidtic and thus will not trigger any overload on the host's immune system. Too, the zeolite may be modified or synthesized in such a way that it cannot adsorb the specific metal cation of the cytochrome enzyme (an iron cation in P450), so no interference would likely occur.

Manifestations of herpes, such as so-called cold sores and fever blisters (H. febrilis), also yield to topical treatment. Phillipsite in the form of wetted powder (_{—}75_m) eliminated such skin infection in a day or less. Furthermore, as an effective desiccant, zeolites quickly dry the sore area, speeding up the healing. Warts of viral origin (e.g., plantar warts) were eliminated likewise in a longer time—about 10-14 days—using wetted clinoptilolite powder incorporated in dressings, or adhesive bandages.

Severe symptoms of shingles (Herpes zoster) were eliminated within a week, healing within the next 14 days virtually without scarring. It is reasonable to assume ion adsorbents to have similar effects on other manifestations of viral infections, e.g., genital herpes, HPV, etc.

This rationale has been implemented successfully in upper respiratory infection symptomatically diagnosed as “common cold” generally considered being of viral origin. Liquid suspensions of zeolite particles in a range from 10 to 75 μm may be used as an inhalant to eliminate difficulty in breathing, and as a gargle to alleviate soreness of the throat. Concentrations of a few weight percent (e.g., 4%) are recommended for the aerosol, and somewhat higher (e.g., 10%) for the gargle.

C. In experiments with fungi (incl. yeasts), only the growth of Saccharomyces cerevisiae and Penicillium notatum was successfully inhibited by all tested species and forms of zeolites (inconclusive results were considered as negative). Virgin chabazite inhibited growth of Achlya spec., Saccharomyces cerevisiae, Penicillium notatum, and Candida kefyr (inconclusive results were considered as negative). Virgin phillipsite inhibited growth of Achlya spec., Saccharomyces cerevisiae, and Penicillium notatum. (inconclusive results were considered as negative).

1. An H⁺ homo-ionized zeolitic composition therapeutic against infection of a mammalian host by member bacteria within at least one category of the Bacillus anthracis Group: viz., B. anthracis, B. cereus, B. mycoides, B. thurigiensis.

2. Natural or synthetic zeolitic composition of claim 1, in colloidal particulate form for application to a mammalian host.

3. Natural or synthetic zeolitic composition of claim 1, having been pretreated in at least one of the following ways:
a. at least partially deionized via equilibration performed by washing such composition with deionized or distilled water;
b. H⁺ homo-ionized by replacing other cations, located at ion-exchanging sites therein, with hydrogen ions to desired extent, e.g., maximum achievable extent.
c. deionized by replacing its H⁺ cations at ion-exchangeable sites therein by equilibration with hyroxyl group anions: OH⁻.

4. Zeolitic composition of claim 3, in colloidal particulate form in at least major part.

5. Zeilitic composition of claim 4, comprising clinoptilolite in at least major part.

6. Zeolitic composition according to claim 1, in finely divided form within or upon a porous cloth or a paper applicator.

7. Zeolitic composition according to claim 1, in the form of an aerosol thereof.

8. Zeolitic composition according to claim 1, in the form of a mist composed in major part of water or similar liquid component.

9. Therapeutic composition comprising a filtrate in the form of a solution/suspension from a filtrate bed composed, in major part, of a natural or synthetic zeolitic composition of claim 1.

10. Natural or synthetic zeolitic composition therapeutic against anthrax, pretreated by being substantially H⁺ homoionized, thereby replacing other cations located at ion-exchanging sites therein with hydrogen cations (H⁺) to a substantial extent.

11. Natural or synthetic zeolitic composition therapeutic against anthrax, pretreated and so substantially deionized by replacing its (H⁺) cations at ion-exchangeable sites therein with hydroxyl group anions (OH⁺) as by equilibration.

12. Therapeutic zeolitic composition according to claim 11, thus deionized to the maximum achievable extent.
